(19) United States
(12) Patent Application Publication
(10) Pub. No.: US 2019/0183527 A1
(43) Pub. Date: Jun. 20, 2019

(54) LAPAROSCOPIC GUIDE

(71) Applicant: MACQUARIE UNIVERSITY, North Ryde (AU)

(72) Inventors: John Anthony Cartmill, Cammeray (AU); Andrew Gilmore, Rose Bay (AU)

(21) Appl. No.: 16/323,036

(22) PCT Filed: Aug. 3, 2017
(86) PCT No.: PCT/AU2017/000159

§ 371 Date: Feb. 4, 2019

(30) Foreign Application Priority Data
Aug. 4, 2016 (AU) 2016903078

(51) Int. Cl. A61B 17/34; A61B 17/3201; A61B 17/32; A61B 90/30

(52) U.S. Cl. CPC A61B 17/3468; A61B 17/3474; A61B 2017/00907; A61B 17/320016; A61B 90/30; A61B 17/3201

(57) ABSTRACT

A laparoscopic guide comprises a hollow body having a longitudinal mid-plane and a cross sectional area that tapers from a proximal end to a distal end thereof. The distal end has a distal end face being oriented substantially perpendicular to the longitudinal mid-plane. The distal end face is adapted for communication with an inlet of a trocar port. The proximal end is adapted for receiving a laparoscopic surgical instrument or the like therein prior to the laparoscopic surgical instrument passing through the trocar port. The proximal end comprises an open end face that is oriented obliquely relative to the distal end face.

### FIELD

The invention relates to a laparoscopic guide for use in the field of laparoscopic surgery and to a method of guiding a laparoscopic surgical instrument or the like.

### BACKGROUND

Laparoscopic surgery is a form of keyhole surgery in which operations are performed at an operation site, generally in the abdominal cavity or pelvic cavity of a patient, via one or more small incisions that are located some distance from the operation site. A laparoscope is used to relay an image of the operation site to a display screen so that the surgeon performing the laparoscopic surgery can view the operation site as he conducts the surgery. The surgeon typically manipulates the laparoscope and a number of laparoscopic surgical instruments, tools or the like, through the incisions for use in performing the surgery at the operation site. The instruments and tools are each engaged through a trocar that is positioned in an incision and which provides a means of holding the instrument or tool in place in the incision and in the abdominal or pelvic cavity. In general, the trocar comprises a trocar port at a proximal end and a cannula extending distally therefrom. During surgery, the trocar port rests above the incision and provides access for the instrument and/or tool into the cannula which extends to the operation site.

Laparoscopic surgery is increasing in popularity due to the advantages it can provide to the patient in terms of a smaller post-surgical physical scar, pain reduction and swifter recovery time in comparison with open surgery. However, the surgeon has to manipulate one or more of the laparoscopic surgical instruments and tools during the surgery, which requires dexterity and precision, particularly when it is required to remove one instrument from the trocar and replace it with another instrument or tool. The surgeon must watch the operation site on a display screen associated with the laparoscope whilst simultaneously removing or engaging a surgical instrument quickly and efficiently. The laparoscopic surgical instruments and tools typically include surgical scissors, forceps and suckers/irrigators that are elongate and narrow in their general shape. The trocar port typically has a small aperture in which the instruments and tools are received. Although it is important for instruments and tools to be inserted in the trocar port in a timely and efficient manner during surgery, it can be difficult to quickly find the small aperture of the trocar port with the instrument or tool. The trocar port also has a tendency to move as the laparoscopic surgical instrument is removed therefrom, meaning that the surgeon must reposition it to continue the surgery. This creates further inefficiency in the surgical procedure.

Laparoscopic techniques are improving, however increasingly challenging cases are being faced by surgeons in terms of body habitus (fat) and pathology (larger, more inflamed cases). There is therefore also a need to improve the ease of surgical procedure and the outcome for challenging cases.

### OBJECT OF INVENTION

It is the object of the present invention to substantially overcome or at least ameliorate one or more of the above disadvantages or to substantially meet the above need.

### SUMMARY OF INVENTION

According to a first aspect of the invention, there is disclosed herein a laparoscopic guide comprising a hollow body having a longitudinal mid-plane and a cross sectional area that tapers from a proximal end to a distal end thereof, the distal end having a distal end face being oriented substantially perpendicular to the longitudinal mid-plane, the distal end face being adapted for communication with an inlet of a trocar port, and the proximal end being adapted for receiving a laparoscopic surgical instrument or the like therein prior to the laparoscopic surgical instrument passing through the trocar port, wherein the proximal end has an open end face that is arranged obliquely relative to the distal end face.

The obliquely oriented open end face is thus advantageously larger than would be the case if the opening was parallel to the distal end face, providing a large area into which the surgical instrument or tool can be engaged. The tapering of the hollow body towards the distal end easily guides the surgical instrument into the trocar and the surgeon is able to manipulate the surgical instrument through a wide range of movement at the proximal open end face to achieve the desired positioning of the surgical instrument at the operation site. Furthermore, the oblique slant of the open end face of the laparoscopic guide maximises access to the trocar without limiting the reach of the surgeon when engaging the surgical instrument into the trocar.

Preferably, the hollow body has a generally conical or part conical shape. In an embodiment, the hollow body is radially symmetrical about a longitudinal axis thereof. In an alternative embodiment, the hollow body is radially asymmetrical about the longitudinal axis.

Preferably, the longitudinal mid-plane is a horizontal mid-plane and an angle subtended by a plane defined by the open end face of the proximal end and the horizontal mid-plane is 45 degrees or less. More preferably, the angle is 30 degrees or less. In an embodiment, the angle is 5 degrees or less.

Preferably, the distal end of the laparoscopic guide is engageable with an inlet portion of a trocar port. In an embodiment, the distal end is engageable by a push fit with the inlet end of the trocar port. Alternately, the distal end is engageable with the inlet of the trocar port via a quick release mechanism, for example a simple releasable clip and recess mechanism.

In an alternative embodiment, the distal end of the laparoscopic guide is formed integrally with an inlet portion of the trocar port.

According to a second aspect of the invention, a method of guiding a laparoscopic surgical instrument or the like into an inlet of a trocar port comprises engaging the laparoscopic surgical instrument or the like into the open end face of the proximal end of a laparoscopic guide as described above and thereafter engaging the laparoscopic surgical instrument or the like into the trocar port.

### BRIEF DESCRIPTION OF DRAWINGS

Preferred embodiments of the present invention will now be described, by way of examples only, with reference to the accompanying drawings, wherein:

FIG. 1 is a schematic cross-sectional representation of a laparoscopic guide attached to a trocar in situ at an operation site;

FIG. 2 is a side view of a typical trocar comprising a trocar port and cannula;

FIG. 3 is a schematic cross-sectional view of the trocar port and cannula of FIG. 2;

FIG. 4 is a schematic enlarged view of the laparoscopic guide fitted to the trocar port of FIG. 2 or 3;

FIG. 5 is an enlarged view of an embodiment of the laparoscopic guide fitted to the trocar port of FIG. 2 or 3 with a push fit attachment;

FIGS. 6a to 6c show schematic cross-sectional views of embodiments of the laparoscopic guide of FIG. 1;

FIG. 7 is an enlarged view of a further embodiment of the laparoscopic guide fitted to the trocar port with a quick release attachment; and

FIG. 8 is a schematic representation of a still further embodiment of a laparoscopic guide that is formed integrally with the trocar port.

### DESCRIPTION OF EMBODIMENTS

Throughout the disclosure herein, as is traditional when referring to relative positioning on a surgical instrument, the term “proximal” refers to the end which is closer to the surgeon and the term “distal” refers to the end which is further away from the surgeon.

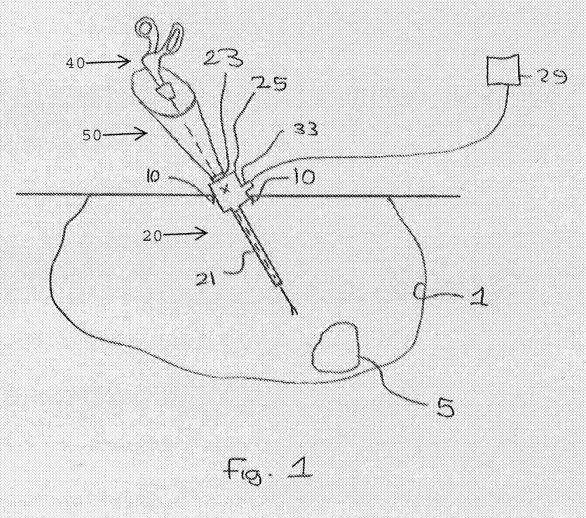

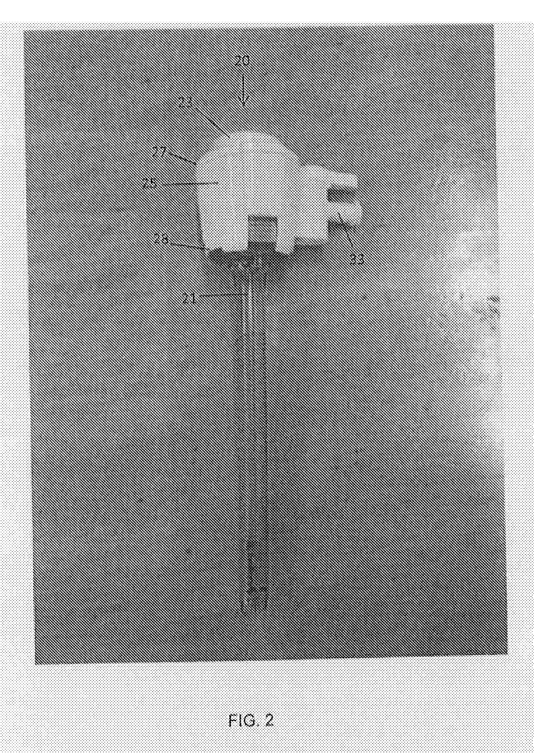

FIG. 1 shows a schematic representation of a laparoscopic surgery taking place at an operation site 5 in the abdominal cavity 1 of a patient. An incision 10 has been made in the patient's abdomen. A trocar 20 is engaged into the abdominal cavity 1 via the incision 10. The trocar 20 has a trocar port 25 at a proximal end thereof and an elongate cannula 21 extending distally therefrom. As shown in FIG. 1, the trocar port 25 rests above the incision 10 and the cannula 21 extends into the abdominal cavity 1. The trocar port 25 is shown more clearly in FIGS. 2 and 3. The trocar port 25 has a plastic body having a central conduit 26 extending from an inlet end 27 to an outlet end 28 thereof. The conduit 26 includes a valve (not shown) therein that is designed to create a seal around a laparoscopic surgical instrument or the like, for example surgical scissors 40, engaged into the trocar port 25 during a surgical procedure. The valve seals the outlet end 28 of the port 25 from the atmosphere during the laparoscopic surgical procedure. The trocar port 25 includes a shallow plastic inlet portion 23 at a proximal end thereof, having a concaved surface 23a that includes an inlet aperture 24 at a centre thereof. The inlet aperture 24 is in communication with the conduit 26 for admission of the surgical scissors 40 or the like into the trocar port 25, through the valve and thereafter into the trocar cannula 21 and into the abdominal cavity 1.

A CO₂ port 33 extends from one side of the trocar port 25. The CO₂ port 33 is in fluid communication with a CO₂ conduit 34 that extends from the CO₂ port 33 and through the plastic body of the trocar port 25 to the outlet end 28 thereof. The CO₂ conduit 34 is also in fluid communication with the trocar cannula 21. During a laparoscopic procedure, a CO₂ supply 29 is connected to the CO₂ port 33 for providing CO₂ to the abdominal cavity 1. The abdominal cavity 1 is insufflated with the carbon dioxide gas in order to maintain the abdominal cavity wall away from the operation site 5. This creates a working and viewing area around the operation site 5 within the abdominal cavity 1, remote from the incision 10. The valve, in sealing the outlet end 28 of the trocar port 25 from the atmosphere, prevents the egress of CO₂ gas from the trocar port 25 to the atmosphere during the laparoscopic surgical procedure.

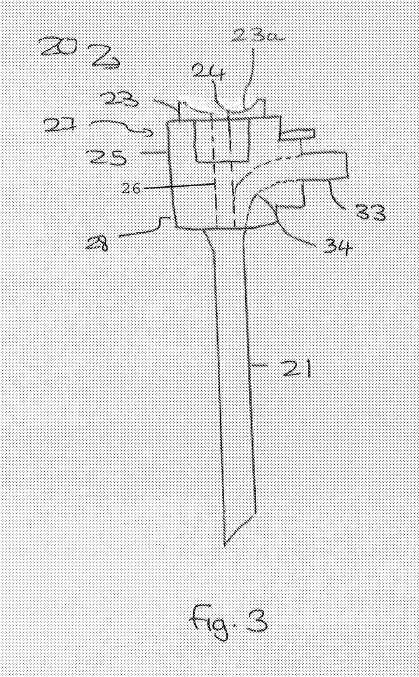

A laparoscopic guide 50 is attached to the inlet portion 23 of the trocar port 25 for guiding the surgical scissors 40 or other surgical instrument or laparoscopic tool into the inlet aperture 24 of the concaved surface 23a of the trocar port 25 and through the cannula 21. In the embodiments of FIGS. 1 and 4 to 8 shown herein, the laparoscopic guide 50 has a generally conical, or funnel, shaped elongate hollow body 52. The hollow body 52 tapers from the proximal end 55 to a distal end 60 thereof. The distal end 60 is cylindrical in shape and is adapted to push fit over the inlet portion 23 of the port 25 for attachment thereto.

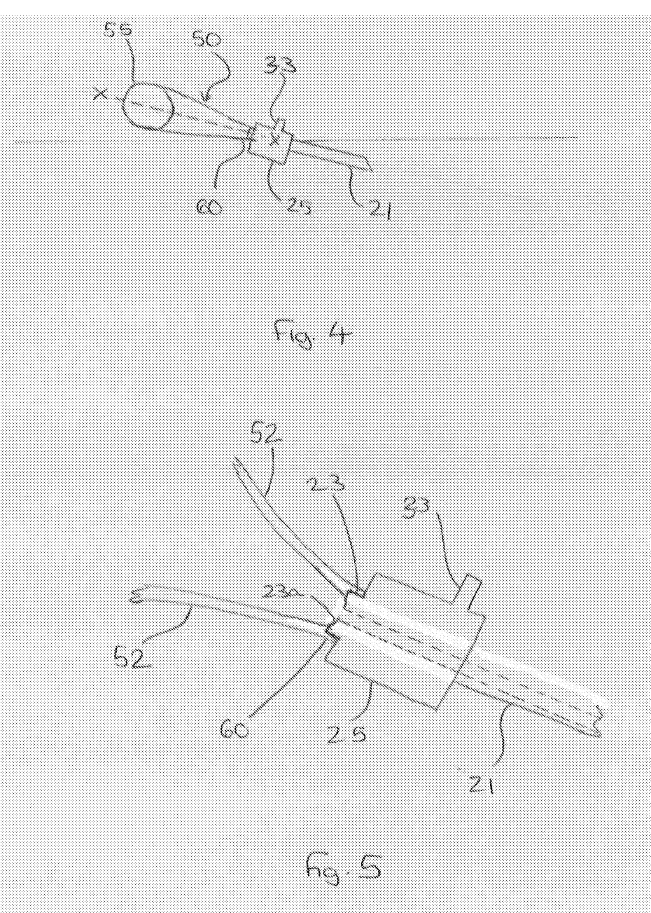

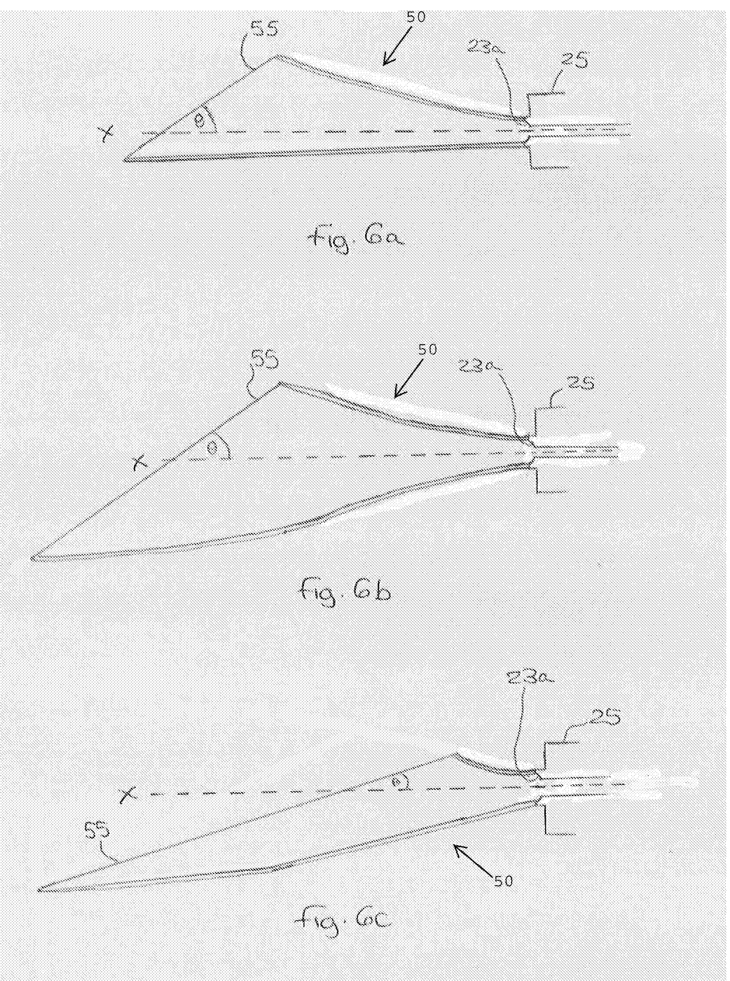

The hollow body 52 flares outwardly towards the proximal end 55 and terminates in an open end face through which the surgical scissors 40 are received during the laparoscopic surgical procedure. The hollow body 52 has a longitudinal mid-plane X-X that extends from the proximal end 55 to the distal end 60 of the hollow body 52 as shown in FIGS. 6a-6c and includes a longitudinal axis of the hollow body 52. The distal end is defined by a distal end face 60 that, in this embodiment, is an open end face. The distal end face 60 is therefore adapted to communicate with the inlet aperture 24 of the trocar port 25. The open end face at the proximal end 55 is arranged obliquely relative to the distal end face 60 of the hollow body, as best seen in FIGS. 6a-6c. In FIGS. 6a-6c, the longitudinal mid-plane is a horizontal mid-plane and an angle θ subtended by a plane defined by the open end face of the proximal end 55 and the horizontal mid-plane that will depend on the requirement of the particular surgery. For example, it is envisaged that the angle θ could be more than 45 degrees in some instances, or it may be very low e.g. 10 degrees or less such that the guide takes the form of a shallow scoop, or any suitable angle in between. The oblique open end face of the proximal end 55 is thus larger than would be the case if the opening was parallel to the distal end face 60. The smaller the angle θ, the larger the opening at the open end face. Consequently, a large area is provided into which the surgical scissors 40 or other instrument or tool can be engaged therein. The tapering of the hollow body 52 towards the distal end 60 quickly guides the surgical scissors into the inlet aperture 24 of the concaved surface 23a of the trocar port 25. The skilled person will note that the surgeon is able to manipulate the surgical scissors 40 through a wide range of movement at the open end face of the proximal end 55 to achieve the desired positioning of the surgical scissors 40 at the operation site 5. Furthermore, the oblique slant of the open end face of the proximal end 55 of the laparoscopic guide 50 maximises access to the trocar port 25 without limiting the reach of the surgeon when engaging the surgical scissors 40 into the trocar port 25.

The ease of insertion of the surgical scissors 40 into the laparoscopic guide 50 and thereby into the trocar port 25 and trocar cannula 21 allows the surgeon to quickly change instruments and tools without spending valuable time aligning the instruments and tools with the inlet aperture 24 of the trocar port 25. The surgical scissors 40 can be engaged and removed from the trocar port 25 and cannula 21 in a stable manner.

In variations of the laparoscopic guide 50, the hollow body 52 may have an asymmetrical cross-sectional area as shown schematically in FIG. 6a or other suitably shaped cross-sectional area without departing from the scope of the claimed invention. Furthermore, a shallower angle θ will provide a larger open end face of the proximal end 55 than will a larger angle θ, improving the reach of the surgeon relative to the trocar port 25. The hollow body 52 can be as long or as short as necessary to stably guide specific surgical instruments into the concaved surface 23a of the trocar port 25.

In other variations of the laparoscopic guide 50, the hollow body 52 may be fully or partially formed from a semi translucent material and internally illuminated by a light source disposed therein such that the surgeon is able to readily locate the hollow body 52 during surgery, especially in the dark. This allows the surgeon to easily insert the surgical scissors 40 or other surgical instrument or laparoscopic tool into the laparoscopic guide 50 and thereby into the trocar port 25 and trocar cannula 21. In other variations of the laparoscopic guide 50, the hollow body 52 may be fully or partially formed from a flexible material.

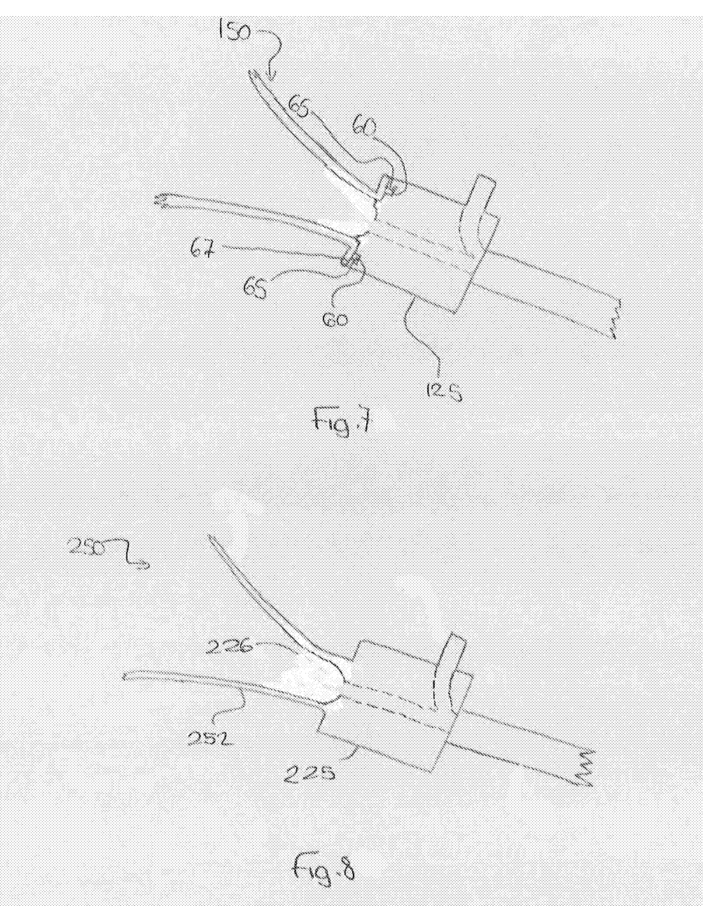

In an alternative embodiment of the laparoscopic guide shown in FIG. 7, the distal end face 60 of the laparoscopic guide 150 has a pair of quick release clips thereon that releasably clip into corresponding recesses 65 arranged in a proximal surface 67 of the trocar port 125. The skilled person will appreciate that other suitable means of attaching the laparoscopic guide 150 to the port 125 are also possible.

In a further alternative embodiment shown in FIG. 8, the laparoscopic guide 250 is formed integrally with the trocar port 225 such that the interior of the hollow body 252 communicates directly with the conduit 226 of the trocar port 225 and the open distal end face 60 is integral with the trocar port 225.

Persons skilled in the art will appreciate that the above specific embodiments described are merely examples of the present disclosure. Persons skilled in the art will appreciate that the various features described in relation to different embodiments may be used in combination or as alternatives. Persons skilled in the art will also appreciate various other modifications and alternatives to the embodiments described.

1. A laparoscopic guide comprising
a hollow body having a longitudinal mid-plane and a cross sectional area that tapers from a proximal end to a distal end thereof,
the distal end having a distal end face being oriented substantially perpendicular to the longitudinal mid-plane, the distal end face being adapted for communication with an inlet of a trocar port,
and the proximal end being adapted for receiving a laparoscopic surgical instrument or the like therein prior to the laparoscopic surgical instrument passing through the trocar port,
wherein the proximal end comprises an open end face that is oriented obliquely relative to the distal end face.

2. The laparoscopic guide of claim 1, wherein the hollow body has a generally conical or part conical shape.

3. The laparoscopic guide of claim 2, wherein the hollow body is radially symmetrical about a longitudinal axis thereof.

4. The laparoscopic guide of claim 2, wherein the hollow body is radially asymmetrical about a longitudinal axis thereof.

5. The laparoscopic guide of any one of claims 1 to 4, wherein an angle subtended by a plane defined by the open end face of the proximal end and the longitudinal mid-plane is 45 degrees or less.

6. The laparoscopic guide of claim 5, wherein the angle is 30 degrees or less.

7. The laparoscopic guide of claim 5 or claim 6, wherein the angle is 10 degrees or less.

8. The laparoscopic guide of any one of claims 1 to 7, wherein the distal end face is engageable with an inlet portion of the trocar port.

9. The laparoscopic guide of claim 8, wherein the distal end face is an open end face that is engageable with the inlet portion of the trocar port via a push fit therewith.

10. The laparoscopic guide of claim 9, wherein the distal end face is engageable with the inlet portion of the trocar port via a quick release mechanism.

11. The laparoscopic guide of claim 10, wherein the quick release mechanism comprises at least one clip associated with either one of the distal end face or the trocar port and at least one recess in the other of the distal end face or the trocar port.

12. The laparoscopic guide of any one of claims 1 to 7, wherein the distal end face is an open end face and wherein the distal end is formed integrally with the inlet portion of the trocar port.

13. The laparoscopic guide of any one of claims 1 to 12, further comprising a light source disposed in the hollow body, and wherein the light source is configured to illuminate the hollow body.

14. The laparoscopic guide of any one of claims 1 to 13, wherein the hollow body is substantially formed from a semi translucent material.

15. The laparoscopic guide of any one of claims 1 to 13, wherein the hollow body is substantially formed from a flexible material.

16. A method of guiding a laparoscopic surgical instrument or the like into an inlet of a trocar port, the method comprising engaging the laparoscopic surgical instrument or the like into the open end face of the proximal end of the laparoscopic guide of any one of claims 1 to 15 and thereafter engaging the laparoscopic surgical instrument or the like into the trocar port.
